(19) United States
(12) Patent Application Publication
(10) Pub. No.: US 2021/0201202 A1
(43) Pub. Date: Jul. 1, 2021

(54) SYSTEM AND METHOD FOR TRAINING A MACHINE LEARNING MODEL BASED ON USER-SELECTED FACTORS

(71) Applicant: KONINKLIJKE PHILIPS N.V., Eindhoven (NL)

(72) Inventor: Eran SIMHON, Boston, MA (US)

(21) Appl. No.: 17/125,308

(22) Filed: Dec. 17, 2020

Related U.S. Application Data
(60) Provisional application No. 62/954,751, filed on Dec. 30, 2019.

(51) Int. Cl. G06N 20/00

(52) U.S. Cl. CPC G06N 20/00; G06F 3/04842

(57) ABSTRACT

In certain embodiments, graphical representations of factors for risk adjustment of a key performance indicator may be presented, and a user selection of a factor subset may be received. Training information may be provided as input to a machine learning model to predict values of the key performance indicator for the selected factor subset. The training information may indicate values of the factor subset associated with a provider. Reference feedback may then be provided to the machine learning model, the reference feedback comprising historic values of the key performance indicator for the provider based on the values of the factor subset that are associated with the provider. The machine learning model may then update portions of the machine learning model based on the reference feedback. The values of the factor subset may then be provided to the updated machine learning model to obtain predicted values of the key performance indicator.

### CROSS-REFERENCE TO PRIOR APPLICATIONS

This application claims the benefit of U.S. Provisional Application No. 62/954,751, filed on 30 Dec. 2019. This application is hereby incorporated by reference herein.

### BACKGROUND

### 1. Field

The present patent application discloses various systems and methods relating to facilitating training or configuration of a prediction model based on user-selected factors.

### 2. Description of the Related Art

Systems and methods for evaluating performance based on key performance indicators are known. The present patent application offers improvements in such systems.

### SUMMARY

Aspects of the invention relate to methods or systems for facilitating training a machine learning model based on user-selected factors. As an example, the machine model may be trained such that the machine learning model is able to predict values of one or more key performance indicators based on values of the user-selected factors.

In some embodiments, graphical representations of factors for risk adjustment of a KPI value may be presented. For example, factor groups (e.g., demographics, chronic conditions, or social determinants of health) may affect KPI values for certain providers. In some embodiments, a user selection of a factor subset, based on the graphical representations, may be received. Training information may then be provided as input to a machine learning model to predict values of the KPI for the selected factor subset. In some embodiments, the training information may indicate values of the factor subset associated with a provider. Reference feedback may then be provided to the machine learning model. In some embodiments, the reference feedback may comprise historic values (e.g., values from the previous year) of the KPI for the provider based on the values of the factor subset that are associated with the provider. In some embodiments, the machine learning model may update one or more portions of the machine learning model based on the reference feedback. Once the machine learning model has updated the portions, the values of the factor subset may be provided to the machine learning model to obtain predicted values of the KPI. The predicted values of the KPI may subsequently be compared to the real values of the KPI to determine a risk adjusted KPI value.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. The mere fact that certain elements are recited in mutually different dependent claims does not indicate that these elements cannot be used in combination.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

FIG. 1 shows an exemplary system for facilitating training or configuration of a prediction model, in accordance with various embodiments;

FIG. 2 shows a graphical user interface and a dataset based on selections of the graphical user interface, in accordance with various embodiments;

FIG. 3 shows graphs of the impacts of various factors on the selected key performance indicator, in accordance with various embodiments;

FIG. 4 shows risk adjusted key performance indicator values for various providers, in accordance with various embodiments; and

FIG. 5 shows a method of facilitating training of a machine learning model based on user-selected factors related to a key performance indicator, in accordance with various embodiments.

### DETAILED DESCRIPTION OF EXEMPLARY EMBODIMENTS

As used herein, the singular form of “a”, “an”, and “the” include plural references unless the context clearly dictates otherwise. As used herein, the term “or” means “and/or” unless the context clearly dictates otherwise. As employed herein, the term “number” shall mean one or an integer greater than one (i.e., a plurality).

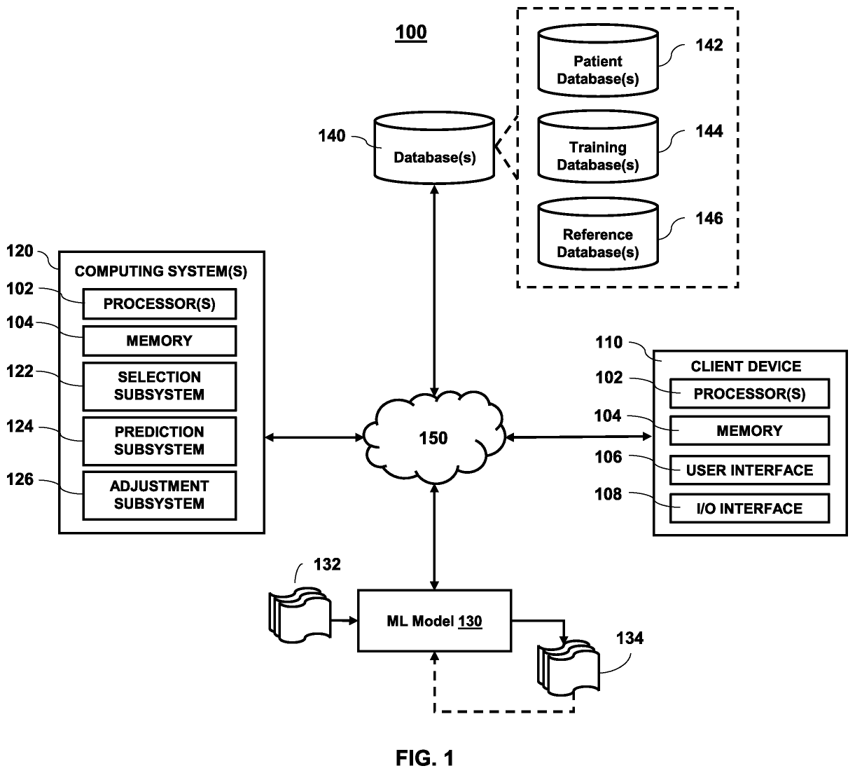

FIG. 1 shows an exemplary system 100 for facilitating training or configuration of a prediction model, in accordance with various embodiments. In some embodiments, system 100 comprises a client device 110, computer system 120 (e.g., one or more servers or other computer systems), machine learning model 130, database(s) 140, and network(s) 150. Although only a single client device 110 is illustrated, system 100 may include multiple client devices that are the same or similar to client device 110. Client device 110, computer system 120, machine learning model 130, and database(s) 140 are configured to be operatively coupled to one another such that each of client device 110, computer system 120, machine learning model 130, and database(s) 140 can communicate with one another, or with other components, devices, and systems, via network 150. For example, network 150 is capable of being accessed by any component of system 100 using Transfer Control Protocol and Internet Protocol (“TCP/IP”) (e.g., any of the protocols used in each of the TCP/IP layers), Hypertext Transfer Protocol (“HTTP”), WebRTC, SIP, and wireless application protocol (“WAP”). In one embodiment, network 150 facilitates communications between components of system 100 or other components with one another via a web browser using HTTP. Various additional communication protocols used to facilitate communications between components of system 100 include, but are not limited to, Wi-Fi, Bluetooth, radio frequency systems (e.g., 900 MHz, 1.4 GHz, and 5.6 GHz communication systems), cellular networks (e.g., GSM, AMPS, GPRS, CDMA, EV-DO, EDGE, 3GSM, DECT, IS-136/TDMA, iDen, LTE or any other suitable cellular network protocol), infrared, BitTorrent, FTP, RTP, RTSP, SSH, or VOIP.

It should be noted that, although some embodiments are described herein with respect to machine learning models, other prediction models (e.g., statistical models or other analytics models) may be used in lieu of or in addition to machine learning models in other embodiments (e.g., a statistical model replacing a machine learning model and a non-statistical model replacing a non-machine-learning model in one or more embodiments).

Client device 110 may include any type of mobile terminal, fixed terminal, or other device. By way of example, client device 110 may include a desktop computer, a notebook computer, a tablet computer, a smartphone, a wearable device, or other client device. Users may, for instance, utilize one or more client device 110 to interact with one another, one or more servers, or other components of system 100. Client device 110 can additionally or alternatively include: a short-range wireless communication module (e.g., a low power 2.4 GHz wireless communication device), an inertial sensor (e.g., an accelerometer and/or gyroscope sensor), an input field (e.g., a touchscreen), a processor, or a rechargeable battery. Client device 110 may include, for example, graphical user interface presented on displays (e.g., a cathode ray tube (CRT) or liquid crystal display (LCD) monitor), pointing devices (e.g., a computer mouse or trackball), keyboards, keypads, touchpads, scanning devices, voice recognition devices, gesture recognition devices, printers, audio speakers, microphones, cameras, or the like. Client device 110 may be connected to computer system 120 from a remote location and may be connected to computer system 120 via network 150. It should be noted that, while one or more operations are described herein as being performed by particular components of computer system 120, those operations may, in some embodiments, be performed by other components of computer system 120 or other components of system 100. As an example, while one or more operations are described herein as being performed by components of computer system 120, those operations may, in some embodiments, be performed by components of client device 110, and vice versa.

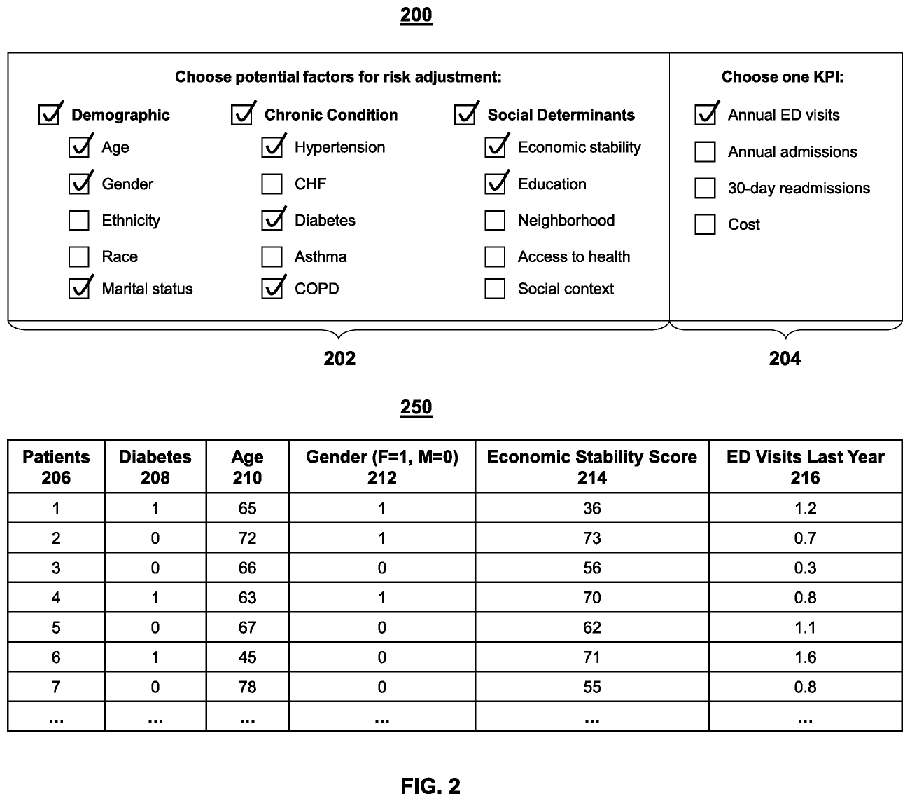

Databases 140 include one or more patient database(s) 142, one or more training database(s) 144, one or more reference database(s) 146, and/or one or more other databases. In some embodiments, patient database 142 contains information about patients. In some embodiments, patient database 142 contains information about providers. In some embodiments, providers may be health care providers, primary care physicians, hospitals, specialists, or other providers. In some embodiments, patient database 142 may include information about chronic conditions, demographics, social determinants, or other patient information (e.g., described in further detail in relation to FIG. 2). In some embodiments, training database 144 may contain training information (e.g., for training a machine learning model such as machine learning model 130). For example, training information may include values of factors in factor groups (e.g., chronic conditions, demographics, social determinants, or other factor groups) associated with providers. In some embodiments, reference database 146 may include reference information (e.g., to be used as feedback for training machine learning model 130). For example, reference information may include historic values (e.g., values from the previous year) of a KPI for providers based on values of factors that are associated with the providers. In some embodiments, a machine learning model (e.g., machine learning model 130) may be training using information from patient database 142 and/or training database 144 and may be updated using information from reference database 146. This example is not intended to be limiting, and various other structures may be used.

In some embodiments, computer system 120 includes a selection subsystem 122, a prediction subsystem 124, and an adjustment subsystem 126. Furthermore, computer system 120 and client device 110 may include one or more processors 102, memory 104, and/or other components. Memory 104 may include computer program instructions that, when executed by processors 102, effectuate operations to be performed, including causing the functions of any of subsystems 122-126 to be performed. The computer program instructions may refer to machine-readable instructions stored within memory 104 and executable by processors 102 automatically, in response to a request to perform a particular function or functions, or both.

In some embodiments, selection subsystem 122 may take user selection input of factors for risk adjustment and KPIs (e.g., via user interface 106). In some embodiments, the factors for risk adjustment may be factors which affect the performance of certain providers with respect to a KPI. In some embodiments, the user may select a set of factors, a subset of factors, a single factor, a factor group, or any other combination of factors. For example, providers who provide care to different age populations, different geographical areas, or patients with different rates of chronic conditions may perform better or worse with respect to certain KPIs. In some embodiments, factor groups may include chronic conditions, demographics, social determinants, or other factors. In some embodiments, the demographic factor group may include age, gender, ethnicity, race, marital status, or other demographic conditions. In some embodiments, the chronic condition factor group may include hypertensions, congestive heart failure (CHF), diabetes, asthma, chronic obstructive pulmonary disease (COPD), or other chronic conditions. In some embodiments, the social determinant factor group may include economic stability, education, neighborhood, access to health, social context, or other social determinant conditions. In some embodiments, KPIs may comprise various metrics of performance of health care providers. For example, KPIs may include annual emergency department (ED) visits, annual hospital admissions, 30-day hospital re-admissions, cost of care, or other KPIs. In some embodiments, selection subsystem 122 may require at least one selection of a factor for risk adjustment and at least one selection of a KPI. In some embodiments, once selection subsystem 122 receives selections of the factor(s) for risk adjustment and of the KPI(s), computing system 120 may retrieve values corresponding to the selected factors (e.g., for the provider(s) under analysis). For example, computer system 120 may retrieve patient information corresponding to the selected factors from patient database 142. In some embodiments, patient information may include values for the selected factors (e.g., gender, age, economic stability score, etc.). Computer system 120 may retrieve this information for further analysis (e.g., as described in further detail below).

In one scenario, with respect to FIG. 2, system 100 may provide a user with a graphical user interface 200. In some embodiments, graphical user interface 200 may comprise various risk adjustment factors 202 and various KPIs 204. Risk adjustment factors 202 and KPIs 204 may vary based on the providers under analysis, factors of interest, KPIs of interest, or other considerations. In some embodiments, client device 110 or computer system 120 may provide the user with graphical user interface 200 via a user interface (e.g., user interface 106 of client device 110, as shown in FIG. 1). The user interface may provide the user with selectable risk adjustment factors and KPIs and may receive input in the form of selections. For example, as shown in FIG. 2, several risk adjustment factors and one KPI have been selected. In some embodiments, computer system 120 may then retrieve information corresponding to the selected factors and KPI for the provider(s) under analysis. For example, computer system 120 may access patient database 142 (e.g., as shown in FIG. 1) in order to retrieve patient data values for patients associated with the providers under analysis. For example, computer system 120 may retrieve patient information corresponding to the selected risk adjustment factors and the selected KPI from patient database 142. Dataset 250 shows a partial data set based on the retrieved patient data from patient database 142. For example, information for patients 206 is shown, including information relating to diabetes 208, age 210, gender 212, and economic stability score 214. Each of patients 206 also shows annual ED visits 216 (i.e., as annual ED visits are the selected KPI). Dataset 250 may show more or less data based on the selected factors of risk adjustment factors 202 and available data in patient database 142. Dataset 250 shows data for some of the selected factors of risk adjustment factors 202, but the additional selected factors of risk adjustment factors 202 (e.g., hypertension, COPD, marital status, and education) may also be shown. In some embodiments, the patient data values (e.g., diabetes 208 values, age 210 values, gender 212 values, economic stability score 214 values, or other patient data values) for patients 206 may be used as training information to train a machine learning model (e.g., as discussed in further detail below).

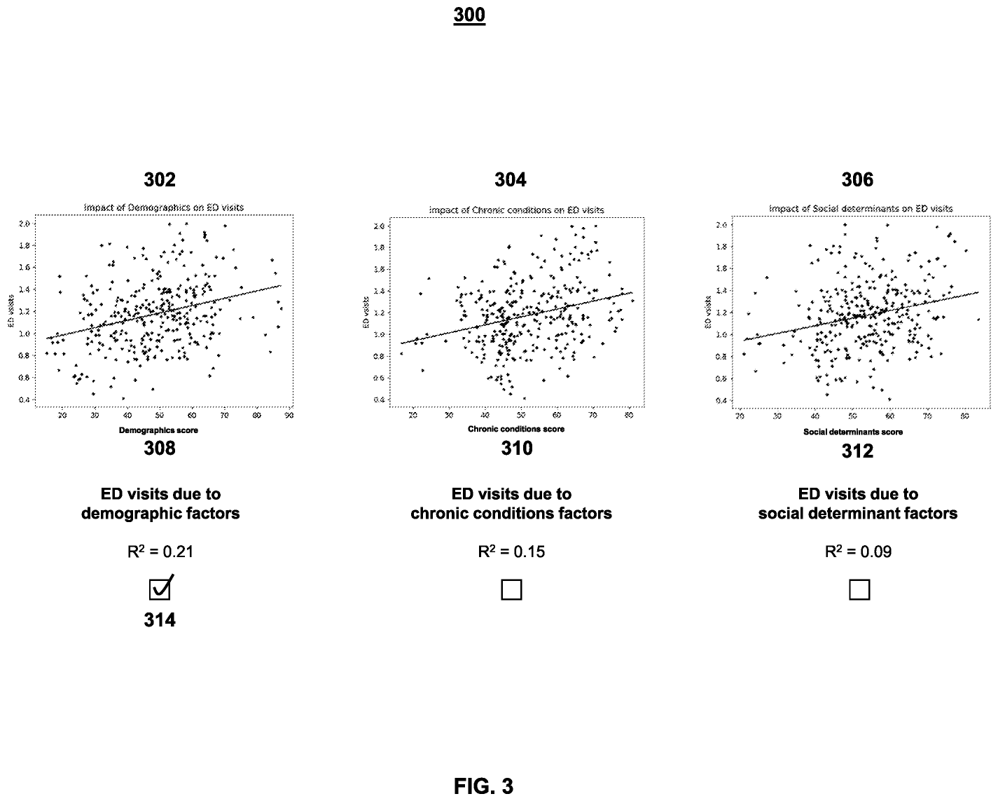

As shown in FIG. 3, KPI values may be displayed with respect to risk adjustment factors as graphs 300. The risk adjustment factors and KPIs may be presented in many different ways, such as charts, graphs, tables, drawings, animations, or other presentation techniques. For example, graphs 302, 304, and 306 show correlations between a set of factors and a selected KPI (e.g., selected from KPIs 204, as shown in FIG. 2). Graph 302 shows a correlation between demographics score 308 and ED visits. Graph 304 shows a correlation between chronic conditions score 310 and ED visits. Graph 306 shows a correlation between social determinants score 312 and ED visits. In graphs 302-306, each data point may represent a patient (e.g., patients 206, as shown in FIG. 2) associated with the provider(s) under analysis. For example, the position of the data point on the respective graph may indicate a value of the set of factors and a value of the KPI, the values being associated with the patient. In some embodiments, the risk adjustment factor data in graphs 302-306 may be based on patient information (e.g., as retrieved from patient database 142, as shown in FIG. 1) indicating risk adjustment factor values. In some embodiments, the KPI data in graphs 302-306 may be based upon historic KPI values (e.g., values from a previous year). For example, the data in graph 302 may be based upon historic KPI values for ED visits based on demographic factors (e.g., age, gender, marital status, or other demographic factors). In some embodiments, the data in graphs 302-306 may be based upon other information sources.

In some embodiments, the KPI data in graphs 302-306 may be based upon predicted KPI values (e.g., as predicted by one or more machine learning models). In some embodiments, a machine learning model or machine learning algorithm may be built and trained for each factor, factor group, factor subset, or other factor combination. For example, the data in graph 302 may be based upon predicted KPI values for ED visits based on demographic factors (e.g., age, gender, marital status, or other demographic factors). In this example, the KPI values may be based upon the outputs of a machine learning model based on the inputs (e.g., demographic factor group). In other examples, a machine learning model may be built for each factor (e.g., age), for multiple factor groups (e.g., for demographic and chronic condition factors), for all selected factors across factor groups, or for a different combination of factors. In some embodiments, a new machine learning model may be built for the factor(s) selected based on graphs 302-306 (e.g., via selection 314), individually or in any combination.

Graphs 302-306 additionally display a regression line based on the data points in each graph. The regression line represents the data of each graph and has an associated R² value. The R² value for each graph indicates the goodness-of-fit of the regression line for each graph 302-306. In other words, the R² value is a measure of how closely the data fits the regression line. Lower values of R² indicate that the regression line does not represent the data well, while higher values of R² indicate that the regression line fits the data well. Graph 302 has the highest R² value with graph 304 having the next highest R² value and graph 306 having the lowest R² value. Therefore, the data in graph 302 is best explained by the regression line.

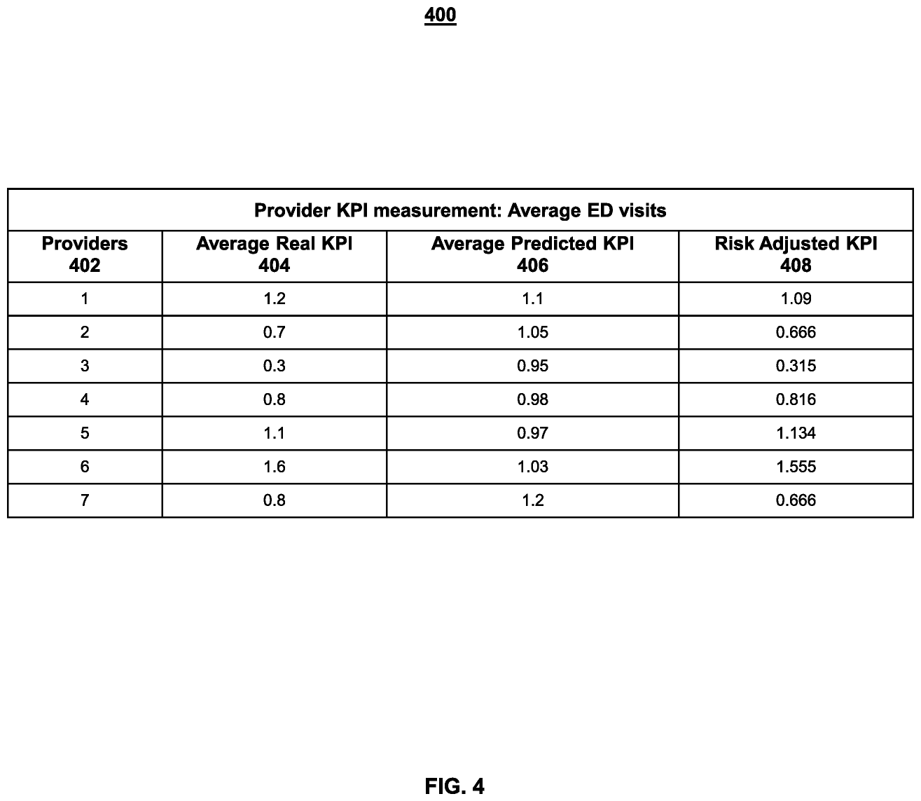

In some embodiments, graphs 302-306 may indicate the impact of the risk adjustment factors on the selected KPI. For example, a graph having a regression line with the steepest slope shows the strongest correlation (i.e., the highest impact) between the risk adjustment factor and the selected KPI. For other types of graphs, other characteristics of the graphs may indicate relative impact of the risk adjustment factors on the KPI. In some embodiments, the risk adjustment factors (e.g., belonging to demographics, chronic conditions, social determinants, or other factor groups) impacting the KPI may be selectable (e.g., as shown in FIG. 3) via selection subsystem 122 of computer system 120 (e.g., as shown in FIG. 1). Therefore, the user may select one or more of the risk adjustment factors based on the associated graph, the impact of the risk adjustment factor on the KPI, and/or some other factor. In some embodiments, computer system 120 may receive the selection via selection subsystem 122. As shown in FIG. 3, graph 302 has been selected (e.g., selection 314). In some embodiments, one or more graphs may be selected. In some embodiments, a selection of a graph (e.g., out of graphs 302, 304, and 306) may represent a selection of the associated factors. In some embodiments, the factors may be selected in some other way. For example, the user may select a machine learning model or algorithm (e.g., as described below). In some embodiments, the risk adjustment factors may be selected before a machine learning model generates predicted KPI values, after a machine learning model generates predicted KPI values, in parallel with a machine learning model generating predicted KPI values, or any combination thereof. In some embodiments, the user may select certain factors at one time during the processing of system 100 and may later select a subset of those factors (e.g., based on graphical representations, predictions, or other factors). In some embodiments, the selected risk adjustment factor(s) (e.g., demographic factors, as shown by selection 314) may then be used to calculate the risk adjusted KPI value (e.g., as discussed in relation to FIG. 4).

Returning to FIG. 1, prediction subsystem 124 of computer system 120 may be used to predict KPI values based on the values of the selected factors (e.g., demographic factors, as described above in relation to FIG. 3). In some embodiments, prediction subsystem 124 may use machine learning model 130 or another prediction model to predict KPI values. In some embodiments, prediction subsystem 124 may communicate with machine learning model 130 or another prediction model. In some embodiments, prediction subsystem 124 may comprise machine learning model 130 or another prediction model. Prediction subsystem 124 and machine learning model 130 are shown as separate entities in FIG. 1, but this example is not intended to be limiting. In some embodiments, machine learning model 130 may combine multiple machine learning models (e.g., using ensemble methods). The use of multiple machine learning models may allow prediction subsystem 124 to generate better predictions.

In some embodiments, machine learning model 130 may include one or more neural networks or other machine learning models. As an example, neural networks may be based on a large collection of neural units (or artificial neurons). Neural networks may loosely mimic the manner in which a biological brain works (e.g., via large clusters of biological neurons connected by axons). Each neural unit of a neural network may be connected with many other neural units of the neural network. Such connections can be enforcing or inhibitory in their effect on the activation state of connected neural units. In some embodiments, each individual neural unit may have a summation function which combines the values of all its inputs together. In some embodiments, each connection (or the neural unit itself) may have a threshold function such that the signal must surpass the threshold before it propagates to other neural units. These neural network systems may be self-learning and trained, rather than explicitly programmed, and can perform significantly better in certain areas of problem solving, as compared to traditional computer programs. In some embodiments, neural networks may include multiple layers (e.g., where a signal path traverses from front layers to back layers). In some embodiments, back propagation techniques may be utilized by the neural networks, where forward stimulation is used to reset weights on the “front” neural units. In some embodiments, stimulation and inhibition for neural networks may be more free flowing, with connections interacting in a more chaotic and complex fashion.

In some embodiments, machine learning model 130 may take inputs 132 and return outputs 134. In some embodiments, inputs 132 may comprise training information indicating values of the selected factors that are associated with the provider(s) under analysis. For example, the training information may comprise patient data values for patients 206 (e.g., as shown in dataset 250 in FIG. 2). Machine learning model 130 may obtain the training information via database(s) 140 (e.g., patient database 142 or training database 144). Machine learning model 130 may use the training information to predict KPI values (e.g., outputs 134). In some embodiments, inputs 132 may further comprise reference feedback. For example, the reference feedback may comprise historic KPI values (e.g., values from a previous year) for the provider(s) under analysis based on the selected risk adjustment factors (e.g., factors selected from risk adjustment factors 202, as shown in FIG. 2, or factors selected by selection 314, as shown in FIG. 3). In some embodiments, machine learning model 130 may obtain the reference feedback from database(s) 140 (e.g., reference database 146). In some embodiments, outputs 134 may comprise predicted KPI values based on the values of the selected factors (e.g., from the training information received as inputs 132).

In some embodiments, machine learning model 130 may assess the predicted KPI values with respect to the reference feedback. For example, machine learning model 130 may compare a predicted KPI value to a KPI value from the reference feedback, both values being based upon the same risk adjustment factor values (e.g., from the training information). If the predicted KPI value does not match the KPI value from the reference feedback, machine learning model 130 may update one or more portions of machine learning model 130. For example, machine learning model 130 may adjust weights, biases, or other parameters of machine learning model 130. In some embodiments, where machine learning model 130 is a neural network, connection weights may be adjusted to reconcile differences between the neural network's prediction and the reference feedback. Some embodiments include one or more neurons (or nodes) of the neural network requiring that their respective errors are sent backward through the neural network to them to facilitate the update process (e.g., backpropagation of error). Updates to the connection weights may, for example, be reflective of the magnitude of error propagated backward after a forward pass has been completed.

In some embodiments, machine learning model 130 may comprise one or more machine learning algorithms. For example, machine learning model 130 may build a machine learning algorithm for each selected factor, each selected factor group, a subset of the factors, or another grouping of factors. In some embodiments, the machine learning algorithms may be linear regression models or another type of model. For example, based on the factors and KPI (e.g., selected from graphical user interface 200 in FIG. 2), machine learning model 130 may determine the following machine learning algorithms for the selected factor groups (demographics, chronic conditions, and social determinants of health, respectively):

### Algorithm 1: Predicted ED Visits=0.01×Age−0.05×Gender+0.2×Marital Status

### Algorithm 2: Predicted ED Visits=0.3×Hypertension+0.2×Diabetes+0.4×COPD

### Algorithm 3: Predicted ED Visits=0.02×Economic Stability+0.01×Education

In some embodiments, as described above, machine learning model 130 may update the machine learning model by adjusting the weights (e.g., coefficients) of the above algorithms based on an assessment of the predictions of machine learning model 130 during training (e.g., based on reference feedback). Once machine learning model 130 has been updated based on the reference feedback, prediction subsystem 124 may utilize the updated machine learning model 130 to predict KPI values based on the patient data values (e.g., as shown in dataset 250 in FIG. 2). For example, prediction subsystem 124 may provide values associated with the selected risk adjustment factors to machine learning model 130. Machine learning model 130 may return one or more predicted values of the KPI based on the values of the selected risk adjustment factors.

In some embodiments, adjustment subsystem 126 of computer system 120 (e.g., as shown in FIG. 1) may determine a risk adjusted KPI value. For example, in some embodiments, adjustment subsystem 126 may obtain real values of the KPI for each patient. In some embodiments, adjustment subsystem 126 may retrieve the real values of KPIs from database(s) 140 (e.g., patient database 142). For example, the real values of the selected KPI may be stored in connection with patients 206 (e.g., annual ED visits 216 in dataset 250, as shown in FIG. 2). In some embodiments, adjustment subsystem 126 may obtain the real KPI values from another source. In some embodiments, adjustment subsystem 126 may compute an average of the real KPI values for a given provider. In some embodiments, adjustment subsystem 126 may compute an average of the predicted KPI values (e.g., as output from machine learning model 130) for the given provider. Adjustment subsystem 126 may then compare the average of the real KPI values with the average of the predicted KPI values. For example, adjustment subsystem 126 may compute a ratio, fraction, percentage, or other comparison between the average of the real KPI values and the average of the predicted KPI values. Adjustment subsystem 126 may then determine the risk adjusted KPI value based on the comparison. For example, in some embodiments, the ratio, faction, or percentage between the average of the real KPI values and the average of the predicted KPI values may be used as the risk adjusted KPI value. In some embodiments, the ratio, faction, or percentage may be used to adjust the average of the real KPI values or the average of the predicted KPI values. In some embodiments, the risk adjusted KPI value may be determined using another method.

In one instance, as shown in dataset 400 of FIG. 4, the average real KPI values 404 and average predicted KPI values 406 are shown for providers 402. In this example, average real KPI values 404 are divided by average predicted KPI values 406, and the resulting number resulting is shown as risk adjusted KPI values 408. Because risk adjusted KPI values 408 comprise a ratio of average real to average predicted KPI values, the risk adjusted KPI values 408 can be evaluated in relation to a threshold of one. If the division of the average real to average predicted KPI values (e.g., values 404 divided by values 406) yields a value of one, then the real KPI values are exactly as predicted (e.g., by machine learning model 130, as shown in FIG. 1) for that provider. In some embodiments, this indicates that the provider performed exactly as expected. In some embodiments, if the division of the average real to average predicted KPI values yields a value less than one, then the real KPI values are, on average, lower than the average predicted KPI values. In cases in which the chosen KPI is better at lower numbers than at higher numbers (e.g., cost), a value of less than one indicates that the provider overperformed (e.g., in relation to the prediction of machine learning model 130). In some embodiments, if the division of the average real to average predicted KPI values yields a value greater than one, then the real KPI values are, on average, higher than the average predicted KPI values. In cases in which the chosen KPI is better at lower numbers than at higher numbers (e.g., cost), a value of greater than one indicates that the provider underperformed (e.g., in relation to the prediction of machine learning model 130). For example, providers 402 having risk adjusted KPI values less than one (e.g., providers 2, 3, 4, and 7) have performed better than expected. For example, for the KPI chosen in FIG. 2, providers 2, 3, 4, and 7 had, on average, fewer ED visits than predicted by machine learning model 130. Providers 402 having risk adjusted KPI values greater than one (e.g., providers 1, 5, and 6) have performed worse than expected. For example, for the KPI chosen in FIG. 2, providers 1, 5, and 6 have had, on average, more ED visits than predicted by machine learning model 130. This example is not intended to be limiting, and risk adjusted KPI values may be evaluated in relation to a different threshold or using a different method.

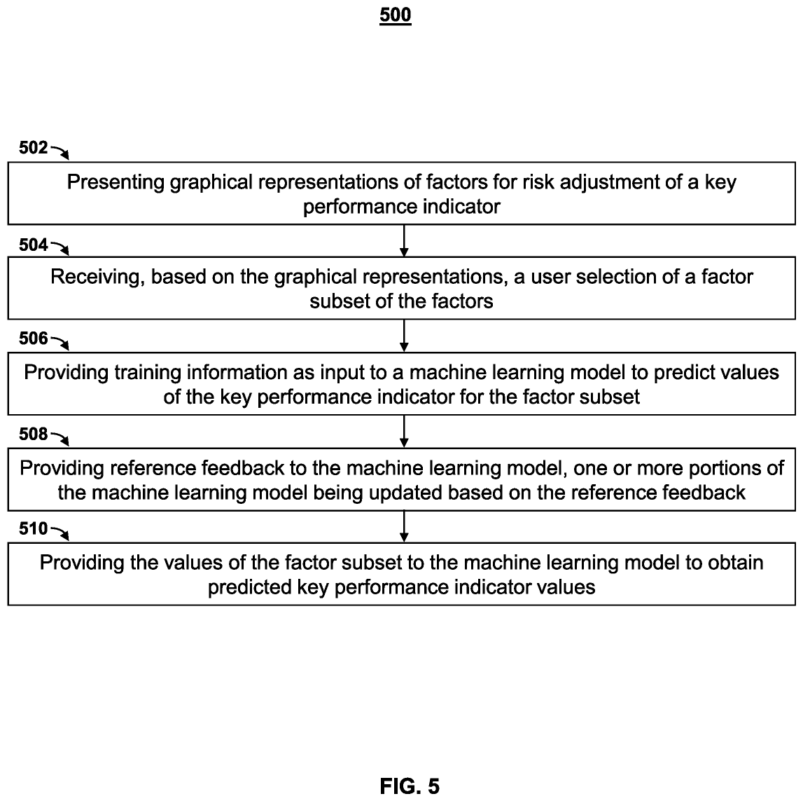

FIG. 5 illustrates method 500 for facilitating training of a machine learning model, in accordance with various embodiments. The operations of method 500 presented below are intended to be illustrative. In some embodiments, method 500 may be accomplished with one or more additional operations not described, and/or without one or more of the operations discussed. Additionally, the order in which the operations of method 500 are illustrated in FIG. 5 and described below is not intended to be limiting.

In some embodiments, method 500 may be implemented in one or more processing devices such as one or more processor(s) 102 of computer system 120 and/or client device 110 (e.g., as shown in FIG. 1). The one or more processing devices may include one or more devices executing some or all of the operations of method 500 in response to instructions stored electronically on an electronic storage medium. The one or more processing devices may include one or more devices configured through hardware, firmware, and/or software to be specifically designed for execution of one or more of the operations of method 500.

At an operation 502, graphical representations of factors for risk adjustment of a KPI are presented. For example, each graphical representations may display an impact of a factor (or set of factors) for risk adjustment on a selected KPI. In some embodiments, graphical representations may represent historic KPI values (e.g., values from the previous year), predicted KPI values, or other KPI values. In some embodiments, operation 502 is performed by a user interface the same as or similar to user interface 106 (shown in FIG. 1 and described herein).

At an operation 504, a user selection of a factor subset is received, based on the graphical representations. In some embodiments, the user selection may be based upon an amount of impact of the factor (or set of factors) on the selected KPI for each graphical representation. In some embodiments, the user selection may be based upon another factor. In some embodiments, operation 504 is performed by a selection subsystem the same as or similar to selection subsystem 122 (shown in FIG. 1 and described herein).

At an operation 506, training information is provided as input to a machine learning model to predict values of the KPI for the factor (or set of factors). In some embodiments, the training information may indicate values of the factor (or set of factors) that are associated with a provider. In some embodiments, operation 506 is performed by a prediction subsystem the same as or similar to prediction subsystem 124 (shown in FIG. 1 and described herein).

At an operation 508, reference feedback is provided to the machine learning model. In some embodiments, the reference feedback may comprise historic values of the KPI for the provider based on the values of the factor (or set of factors). In some embodiments, the machine learning model may perform an assessment of its predicted values (e.g., as determined at operation 506) based on the reference feedback. In some embodiments, one or more portions of the machine learning model are updated based on the reference feedback and/or the assessment. In some embodiments, operation 508 is performed by a prediction subsystem the same as or similar to prediction subsystem 124 (shown in FIG. 1 and described herein).

At an operation 510, values of the factor (or set of factors) are provided to the machine learning model to obtain predicted values of the KPI. In some embodiments, operation 510 is performed only after one or more portions of the machine learning model have been updated (e.g., as described at operation 508). In some embodiments, operation 510 is performed by a prediction subsystem the same as or similar to prediction subsystem 124 (shown in FIG. 1 and described herein).

Returning to FIG. 1, computer system 120 and/or client device 110 may be configured to include one or more processors (e.g., processor(s) 102) coupled to memory 104 and a network 150 via an I/O interface 108. As described herein, a processor can include a single processor or a plurality of processors (e.g., distributed processors). A processor may be any suitable processor capable of executing or otherwise performing instructions. A processor may include a central processing unit (CPU) that carries out program instructions to perform the arithmetical, logical, and input/output operations of computer system 120. A processor may execute code (e.g., processor firmware, a protocol stack, a database management system, an operating system, or a combination thereof) that creates an execution environment for program instructions. A processor may include a programmable processor. A processor may include general or special purpose microprocessors. A processor may receive computer program instructions and data from a memory (e.g., memory 104). Computer system 120 may be a uni-processor system including one processor (e.g., processor 102), or a multi-processor system including any number of suitable processors (e.g., processor(s) 102). Multiple processors may be employed to provide for parallel or sequential execution of one or more portions of the techniques described herein. Processes, such as logic flows, described herein are capable of being performed by one or more programmable processors executing one or more computer programs to perform functions by operating on input data and generating corresponding output. Processes described herein may be performed by, and apparatus can also be implemented as, special purpose logic circuitry, e.g., an FPGA (field programmable gate array) or an ASIC (application specific integrated circuit). Computer system 120 may include a plurality of computing devices (e.g., distributed computer systems) to implement various processing functions.

I/O interface 108 is configured to provide an interface for connection of one or more I/O devices, such as client device 110 to computer system 120. I/O devices may include devices that receive input (e.g., from a patient or provider) or output information (e.g., to a user or provider). I/O interface may be configured to coordinate I/O traffic between processor(s) 102, memory 104, network 150, and/or other peripheral devices. I/O interface 108 may perform protocol, timing, or other data transformations to convert data signals from one component (e.g., memory 104) into a format suitable for use by another component (e.g., processor(s) 102). I/O interface 108 may include support for devices attached through various types of peripheral buses, such as a variant of the Peripheral Component Interconnect (PCI) bus standard or the Universal Serial Bus (USB) standard.

Network 150 may include a network adapter that provides for connection of client device 110 and computer system 120 to a network. Network 150 may facilitate data exchange between computer system 120 and other devices connected to the network. Network 150 may support wired or wireless communication. The network may include an electronic communication network, such as the Internet, a local area network (LAN), a wide area network (WAN), a cellular communications network, or the like.

System memory 104 may be configured to store computer program instructions and/or data. Computer program instructions may be executable by a processor (e.g., one or more of processor(s) 102) to implement one or more embodiments of the present patent application's techniques. Computer program instructions may include modules of computer program instructions for implementing one or more techniques described herein with regard to various processing modules. Computer program instructions may include a computer program (which in certain forms is known as a program, software, software application, script, or code). A computer program may be written in a programming language, including compiled or interpreted languages, or declarative or procedural languages. A computer program may include a unit suitable for use in a computing environment, including as a stand-alone program, a module, a component, or a subroutine. A computer program may or may not correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one or more computer processors located locally at one site or distributed across multiple remote sites and interconnected by a communication network.

Memory 104 may include a tangible program carrier having program instructions stored thereon. A tangible program carrier may include a non-transitory computer readable storage medium. A non-transitory, computer readable medium may include a machine-readable storage device, a machine-readable storage substrate, a memory device, or any combination thereof. The non-transitory, computer readable medium may include non-volatile memory (e.g., flash memory, ROM, PROM, EPROM, EEPROM memory), volatile memory (e.g., random access memory (RAM), static random access memory (SRAM), synchronous dynamic RAM (SDRAM)), bulk storage memory (e.g., CD-ROM and/or DVD-ROM, hard-drives), or the like. Memory 104 may include a non-transitory, computer readable storage medium that may have program instructions stored thereon that are executable by a computer processor (e.g., one or more of processor(s) 102) to cause the subject matter and the functional operations described herein. A memory (e.g., memory 104) may include a single memory device and/or a plurality of memory devices (e.g., distributed memory devices). Instructions or other program code to provide the functionality described herein may be stored on a tangible, non-transitory computer readable media. In some cases, the entire set of instructions may be stored concurrently on the media, or in some cases, different parts of the instructions may be stored on the same media at different times.

Embodiments of the techniques described herein may be implemented using a single instance of client device 110 or computer system 120. Embodiments of the techniques described herein may be implemented using multiple client devices 110 or multiple computer systems 120, each configured to host different portions or instances of embodiments. Multiple client devices 110 or computer systems 120 may provide for parallel or sequential processing/execution of one or more portions of the techniques described herein.

Those skilled in the art will appreciate that system 100 is merely illustrative and is not intended to limit the scope of the techniques described herein. System 100 may include any combination of devices or software that may perform or otherwise provide for the performance of the techniques described herein. For example, client device 110 and computer system 120 may include or be a combination of a cloud-computing system, a data center, a server rack, a server, a virtual server, a desktop computer, a laptop computer, a tablet computer, a server device, a client device, a mobile telephone, a personal digital assistant (PDA), a mobile audio or video player, a game console, a vehicle-mounted computer, or a Global Positioning System (GPS), or the like. Client device 110 and computer system 120 may also be connected to other devices that are not illustrated or may operate as a stand-alone device/system. In addition, the functionality provided by the illustrated components may in some embodiments be combined in fewer components or distributed in additional components. Similarly, in some embodiments, the functionality of some of the illustrated components may not be provided or other additional functionality may be available.

Those skilled in the art will also appreciate that while various items are illustrated as being stored in memory or on storage while being used, these items or portions of them may be transferred between memory and other storage devices for purposes of memory management and data integrity. Alternatively, in other embodiments some or all of the software components may execute in memory on another device and communicate with the illustrated computer system via inter-computer communication. Some or all of the system components or data structures may also be stored (e.g., as instructions or structured data) on a computer-accessible medium or a portable article to be read by an appropriate drive, various examples of which are described above. In some embodiments, instructions stored on a computer-accessible medium separate from computer system 120 may be transmitted to computer system 120 via transmission media or signals such as electrical, electromagnetic, or digital signals, conveyed via a communication medium such as a network or a wireless link. Various embodiments may further include receiving, sending, or storing instructions or data implemented in accordance with the foregoing description upon a computer-accessible medium. Accordingly, the present techniques may be practiced with other computer system configurations.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present patent application contemplates that, to the extent possible, one or more features of any embodiment can be combined with one or more features of any other embodiment.

The present techniques will be better understood with reference to the following enumerated embodiments:

1. A method comprising: providing training information as input to a prediction model to predict values of the key performance indicator for a factor subset, the training information indicating values of the factor subset that are associated with a provider; providing reference feedback to the prediction model, the reference feedback comprising historic key performance indicator values for the provider based on the values of the factor subset that are associated with the provider, the prediction model updating one or more portions of the prediction model based on the reference feedback; subsequent to the updating of the prediction model, providing the values of the factor subset that are associated with the provider to the prediction model to obtain predicted key performance indicator values for the provider.
2. The method of embodiment 1, further comprising: presenting, via a user interface, graphical representations of factors for risk adjustment of a key performance indicator; and receiving, via the user interface, based on the graphical representations, a user selection of a factor subset of the factors.
3. The method of any of embodiments 1-2, further comprising: obtaining real key performance indicator values for the factor subset; comparing an average of the real key performance indicator values to an average of the predicted key performance indicator values for the factor subset; and determining a risk adjusted key performance indicator value based on the comparing.
4. The method of embodiment 3, wherein comparing the real key performance indicator values to the predicted key performance indicator values comprises calculating a ratio of an average of the real key performance indicator values to an average of the predicted key performance indicator values.
5. The method of embodiment 4, further comprising: upon a condition in which the ratio is greater than a threshold, determining that the provider has underperformed; and upon a condition in which the ratio is less than the threshold, determining that the provider has overperformed.
6. The method of any of embodiments 1-5, further comprising receiving a selection indicating the key performance indicator and the factors for risk adjustment of the key performance indicator, and wherein the graphical representations are presented based on the received selection.
7. The method of any of embodiments 1-6, wherein the graphical representations indicate an amount of impact of each factor of the factors on the key performance indicator for the provider.
8. The method of embodiment 7, wherein the user selection of the factor subset is based upon the amount of impact of each factor on the key performance indicator for the provider.
9. The method of any of embodiments 1-8, further comprising comparing the risk adjusted key performance indicator value for the provider to risk adjusted key performance indicator values for other providers.
10. The method of any of embodiments 1-9, wherein the prediction model comprises a neural network or other machine learning model.
11. A non-transitory, machine-readable medium storing instructions that, when executed by a data processing apparatus, causes the data processing apparatus to perform operations comprising those of any of embodiments 1-10.
12. A system comprising: one or more processors; and memory storing instructions that, when executed by the processors, cause the processors to effectuate operations comprising those of any of embodiments 1-10.

What is claimed is:

1. A system for facilitating training of a machine learning model based on user-selected factors related to a key performance indicator, the system comprising:
a computer system that comprises one or more processors programed with computer program instructions that, when executed, cause the computer system to:
present, via a user interface, graphical representations of factors for risk adjustment of a key performance indicator and an amount of impact of each factor of the factors on the key performance indicator for a provider;
receive, via the user interface, based on the presentation of the graphical representations, a user selection of a factor subset of the factors;
obtain, based on the user selection of the factor subset, training information for each factor of the factor subset, the training information comprising datasets indicating values of each factor of the factor subset that are associated with the provider;
provide the training information as input to a machine learning model to predict values of the key performance indicator for each factor of the factor subset;
provide reference feedback to the machine learning model, the reference feedback comprising historic values of the key performance indicator for the provider that occurred in connection with the values of the factor subset associated with the provider, the machine learning model assessing the predicted values of the key performance indicator based on the reference feedback and updating one or more portions of the machine learning model based on the assessment of the machine learning model; and
subsequent to the updating of the machine learning model, provide a first value of the factor subset to the machine learning model to obtain a first predicted key performance indicator value for the provider.

2. The system of claim 1, wherein the computer system is further caused to:
provide a second value of the factor subset to the machine learning model to obtain a second predicted key performance indicator value; and
compute an average predicted key performance indicator value based on the first predicted key performance indicator value and the second predicted key performance indicator value.

3. The system of claim 2, wherein the computer system is further caused to:
obtain an average real key performance indicator value based on a first real key performance indicator value for the factor subset and a second real key performance indicator value for the factor subset;
compare the average predicted key performance indicator value to the average real key performance indicator value; and
determine a risk adjusted key performance indicator value for the factor subset based on the comparing.

4. The system of claim 1, wherein the computer system is further caused to receive a selection indicating the key performance indicator and the factors for risk adjustment of the key performance indicator, and
wherein the graphical representations are presented based on the received selection.

5. A method implemented by one or more processors executing computer program instructions that, when executed, perform the method, the method comprising:
presenting, via a user interface, graphical representations of factors for risk adjustment of a key performance indicator;
receiving, via the user interface, based on the graphical representations, a user selection of a factor subset of the factors;
providing training information as input to a machine learning model to predict values of the key performance indicator for the factor subset, the training information indicating values of the factor subset that are associated with a provider;
providing reference feedback to the machine learning model, the reference feedback comprising historic key performance indicator values for the provider based on the values of the factor subset that are associated with the provider, the machine learning model updating one or more portions of the machine learning model based on the reference feedback;
subsequent to the updating of the machine learning model, providing the values of the factor subset that are associated with the provider to the machine learning model to obtain predicted key performance indicator values for the provider.

6. The method of claim 5, further comprising:
obtaining real key performance indicator values for the factor subset;
comparing an average of the real key performance indicator values to an average of the predicted key performance indicator values for the factor subset; and
determining a risk adjusted key performance indicator value based on the comparing.

7. The method of claim 6, wherein comparing the average of the real key performance indicator values to the average of the predicted key performance indicator values comprises calculating a ratio of the average of the real key performance indicator values to the average of the predicted key performance indicator values.

8. The method of claim 7, further comprising:
upon a condition in which the ratio is greater than a threshold, determining that the provider has underperformed; and
upon a condition in which the ratio is less than the threshold, determining that the provider has overperformed.

9. The method of claim 5, further comprising receiving a selection indicating the key performance indicator and the factors for risk adjustment of the key performance indicator, and
wherein the graphical representations are presented based on the received selection.

10. The method of claim 5, wherein the graphical representations indicate an amount of impact of each factor of the factors on the key performance indicator.

11. The method of claim 10, wherein the user selection of the factor subset is based upon the amount of impact of each factor on the key performance indicator.

12. The method of claim 6, further comprising comparing the risk adjusted key performance indicator value for the provider to risk adjusted key performance indicator values for other providers.

13. A non-transitory, computer-readable medium storing instructions that, when executed by one or more processors, cause operations comprising:
presenting, via a user interface, graphical representations of factors for risk adjustment of a key performance indicator;
receiving, via the user interface, based on the graphical representations, a user selection of a factor subset of the factors;
providing training information as input to a machine learning model to predict values of the key performance indicator for the factor subset, the training information indicating values of the factor subset that are associated with a provider;
providing reference feedback to the machine learning model, the reference feedback comprising historic key performance indicator values for the provider based on the values of the factor subset that are associated with the provider, the machine learning model updating one or more portions of the machine learning model based on the reference feedback;
subsequent to the updating of the machine learning model, providing the values of the factor subset that are associated with the provider to the machine learning model to obtain predicted key performance indicator values for the provider.

14. The non-transitory, computer-readable medium of claim 13, wherein the operations further comprise:
obtaining real key performance indicator values for the factor subset;
comparing an average of the real key performance indicator values to an average of the predicted key performance indicator values for the factor subset; and
determining a risk adjusted key performance indicator value based on the comparing.

15. The non-transitory, computer-readable medium of claim 14, wherein comparing the average of the real key performance indicator values to the average of the predicted key performance indicator values comprises calculating a ratio of the average of the real key performance indicator values to the average of the predicted key performance indicator values.

16. The non-transitory, computer-readable medium of claim 15, wherein the operations further comprise:
upon a condition in which the ratio is greater than a threshold, determining that the provider has underperformed; and
upon a condition in which the ratio is less than the threshold, determining that the provider has overperformed.

17. The non-transitory, computer-readable medium of claim 13, wherein the operations further comprise receiving a selection indicating the key performance indicator and the factors for risk adjustment of the key performance indicator, and
wherein the graphical representations are presented based on the received selection.

18. The non-transitory, computer-readable medium of claim 13, wherein the graphical representations indicate an amount of impact of each factor of the factors on the key performance indicator.

19. The non-transitory, computer-readable medium of claim 18, wherein the user selection of the factor subset is based upon the amount of impact of each factor on the key performance indicator.

20. The non-transitory, computer-readable medium of claim 14, wherein the operations further comprise comparing the risk adjusted key performance indicator value for the provider to risk adjusted key performance indicator values for other providers.
